(19) United States
(12) Patent Application Publication
(10) Pub. No.: US 2020/0269010 A1
(43) Pub. Date: Aug. 27, 2020

(54) TENSILE-STRENGTH-ENHANCING TUBE FOR AN IMPLANTABLE ELECTRODE LEAD OR A CATHETER, ELECTRODE LEAD WITH A TENSILE-STRENGTH-ENHANCING TUBE, AND CATHETER WITH A TENSILE-STRENGTH-ENHANCING TUBE

(71) Applicant: BIOTRONIK SE & Co. KG, Berlin (DE)

(72) Inventors: Detmar Jadwizak, Erkner (DE); Dajana Kaiser, Berlin (DE); Carsten Fruendt, Berlin (DE); Gordon Hillebrand, Berlin (DE)

(21) Appl. No.: 16/795,832

(22) Filed: Feb. 20, 2020

(30) Foreign Application Priority Data
Feb. 25, 2019 (DE) 10 2019 104 641.6

(51) Int. Cl. A61M 25/00; A61N 1/05; A61L 29/04; A61L 29/12

(52) U.S. Cl. CPC A61M 25/005; A61N 1/05; A61M 2205/0233; A61L 29/126; A61M 2025/0048; A61L 29/042

(57) ABSTRACT

A tensile-force-enhancing tube for an implantable electrode lead or a catheter includes a tubular braid which is embedded in an elastomer material, wherein the braid comprises at least one cross thread and at least one axial thread.

### CROSS-REFERENCE TO RELATED APPLICATIONS

This patent application claims the benefit of and priority to co-pending German Patent Application No. DE 10 2019 104 641.6, filed Feb. 25, 2019 in the German Patent Office, which is hereby incorporated by reference in its entirety.

### TECHNICAL FIELD

The present invention relates to a tensile-strength-enhancing tube for an implantable electrode lead or a catheter. In addition, the present invention relates to an electrode lead having a tensile-strength-enhancing tube of this kind, and to a catheter comprising a tensile-strength-enhancing tube of this kind.

### BACKGROUND

Implantable electrode leads are used, for example, for cardiac resynchronization therapy (CRT). Such electrode leads normally have an elongate lead body and are usually connectable in a proximal region to a pulse generator by means of a connection device, such as a plug. In a distal region, such electrode leads have electrodes usually connected to the lead body for the contacting of bodily tissue, for example in the region of the heart. In the case of CRT electrode leads, the distal electrodes are often configured in the form of ring electrodes. Some conventional CRT electrode leads, for example, comprise, in their interior, an electrically conductive “coradial coil”, which is encased externally with silicone for electrical insulation.

At the time of implantation, repositioning, or removal (explantation) of such electrode leads, they are exposed to certain tensile forces. For example, it is thus necessary that an electrode lead must be able to transmit a tensile force of 5 N between its most proximal and most distal ends without sustaining any damage (EIN EN 45502-2-1). For this reason, it is known per se to provide measures for enhancing the tensile strength at least at portions of an electrode lead, for example, in the region of one or more distal ring electrodes.

Various approaches already exist for solving the problem of enhancing the tensile strength of an electrode lead. For example, in a CRT electrode known in the prior art the tensile strength is enhanced by a coil, a polyimide tube in an inner lumen of the coil, a clamping of the coil, and an outer silicone tube for isolation. By way of a sandwich structure of this kind, a sufficient tensile force transmission may be attained with a comparatively small outer diameter of less than 5 F. In addition, the silicone material enables a very good performance during the implantation and over the entire service life of an electrode.

A disadvantage of this solution is that the described sandwich structure is comparatively complex and requires many assembly steps in which many components have to be assembled within a confined installation space, which may lead to waste and high manufacturing costs. The tensile force transmission in this solution is dependent on the clamping of the coil with the inner polyimide tube. An undesirable increase in rigidity may thus result.

A further known approach for solving this problem is based on a special silicone-polyimide material compound which is connected to a polyurethane insulation that is stable under tensile force.

In another solution, an electrode lead comprises multi-lumen tubes made of a special silicone-polyurethane compound with inner cables which transmit the tensile force independently of the hardness of the insulation materials.

Such solutions are comparatively complex and may be associated with high development and production costs since they are based on special material compounds.

Similar challenges with regard to a tensile strength enhancement as described above with reference to electrode leads are also encountered in other medical devices, for example, catheters, which likewise must be able to transmit certain tensile forces and at the same time must have sufficient flexibility in respect of bending stress.

The present invention is directed at overcoming one or more of the above-mentioned problems.

### SUMMARY

On this basis, an object of the present invention is to provide a tensile strength enhancement for an implantable electrode lead or a catheter which, also with a relatively small outer diameter of the electrode lead or of the catheter, ensures a sufficient tensile force transmission and at the same time sufficient flexibility in respect of bending stress. A fatigue strength in respect of mechanical stresses (in particular, with regard to the tensile strength and flexural strength), as is necessary, for example, for permanently implantable electrode leads, should also be ensured. In addition, the solution according to the present invention should enable a facilitated and quicker assembly as compared to known solution approaches. An implantable electrode lead should also be provided with a tensile strength enhancement of this kind and a catheter having a strength enhancement of this kind.

In accordance with a first aspect of the present invention, at least this object is achieved by a tensile-strength-enhancing tube for an implantable electrode lead or a catheter. The tensile-strength-enhancing tube comprises a tubular braid which is embedded in an elastomer material. The braid comprises at least one cross thread and at least one axial thread. The at least one axial thread fundamentally ensures the (axial) tensile force transmission, whereas the braid with the at least one cross thread enables a good anchoring of the at least one axial thread in the elastomer material. For example, the tensile-strength-enhancing tube may to this end also comprise a plurality of axial threads, for example one to ten axial threads. In addition, the tubular braid together with the elastomer material may ensure a good stability of the tensile-force-enhancing tube (and thus the electrode lead) in respect of a radial expansion. In combination with the at least one axial thread, the extensibility of the tensile-strength-enhancing tube/the electrode lead may thus be effectively limited in all directions.

For example, in accordance with one embodiment, only a single cross thread may be provided, which is arranged helically and thus forms the basic form of the tubular braid. In particular, the cross thread defines a radial limitation of the tubular braid. One or more axial threads may be intertwined/interwoven with the cross thread and are intended to transmit the axial tensile forced in the electrode lead.

If the braid is embedded in the elastomer material, on the one hand a sufficient tensile force transmission is thus made possible by the at least one axial thread, and on the other hand the connection of the at least one axial thread to the at least one cross thread at corresponding intersection points prevents the at least one axial thread from being pulled out from the elastomer material under tensile load. On the whole, a more flexible tube which at the same time is more stable under tensile force and may also be produced with a small outer diameter and wall thicknesses is thus provided.

In accordance with one embodiment, the at least one cross thread and the at least one axial thread are woven with one another (i.e., for example the axial thread is guided through alternately above and below the cross thread) or are otherwise fastened to one another at their intersection points, for example by gluing or welding. The stability of the braid may thus be further increased on the whole, and the assembly may be facilitated.

It also lies within the scope of the present invention that an outer diameter of the tensile-strength-enhancing tube may be less than or equal to 5 F. Here, F (“French”) denotes a unit that is conventional within the field of medicine and is used, for example, for cannula and catheter diameters. One French corresponds to ⅓ mm. With a small outer diameter of this kind the tensile-strength-enhancing tube is suitable, for example, for typical CRT electrode leads, which, for example, have a diameter of approximately 1.6 mm (4.8 F) so that they may be introduced into blood vessels having diameters of approximately 1.8 mm to 5 mm.

A wall thickness of the tensile-strength-enhancing tube in accordance with one embodiment may be less than or equal to 0.15 mm. In particular due to a relatively small wall thickness of this kind, small outer diameters, for example, in the above-mentioned range may be attained, such that, with a tensile-strength-enhancing tube according to the present invention, for example, a 4.8 F electrode lead may be realized. A small wall thickness of this kind may also contribute to a particularly good flexibility in respect of bending stress.

In a preferred embodiment, the tubular braid comprises at least two, for example 3 to 12, cross threads. By providing a plurality of cross threads, which might be woven with one another and with the at least one axial thread, the stability of the braid and consequently of the tensile-strength-enhancing tube as a whole is further increased.

In accordance with one embodiment, the elastomer material in which the braid is embedded comprises a silicone or consists of silicone. For example, the braid may be overmolded with liquid silicone rubber (what is known as an LSR compound). Due to the choice of silicone as elastomer material, all known advantages of that material, which has become established within the field of medical engineering, may also be incurred. This relates, for example, to the good extensibility and resultant flexibility in respect of bending stress. In addition, silicone is biocompatible and relatively easily processed. Furthermore, the silicone-based tensile-strength-enhancing tube may be easily adhesively bonded to adjacent silicone components, for example, silicone insulation tubes or injection-molded silicone parts. A silicone-based solution additionally enables an advantageous dimensional variability: The tensile-strength-enhancing tube for example may be produced as a simple tube or as an injection-molded part with more or less complex outer and/or inner contours. This enables use within a wide variety of areas of electrodes and catheters. A further advantage of the use of silicone is its electrically insulating property. The tensile-strength-enhancing tube may thus be used simultaneously as electrical insulation for an electrical lead guided therein. Alternatively to silicone, other elastomers, rubber, or very soft plastics may be used.

In accordance with one embodiment, the at least one cross thread and/or the at least one axial thread comprises a thermoplastic material. For example, the at least one cross thread and/or the at least one axial thread may consist of a thermoplastic material, such as polyurethane (PU), polypropylene (PP), polyamide (PA), or polyethylene terephthalate (PET). Such materials generally may be processed economically and easily and have the necessary mechanical properties in order to—in the case of the axial threads—sufficiently withstand tensile forces or—in the case of the cross threads—largely prevent a radial extensibility of the tensile-strength-enhancing tube. A material combination of PET threads and silicone as elastomer material enables a processing or a use within high temperature ranges, since the melting point of PET is approximately 250° C. and the vulcanization temperature of silicone is approximately 200° C.

In a preferred variant, it is provided that the at least one cross thread and/or the at least one axial thread is a multi-filament thread which is formed of a number of individual threads. It may also be provided that the elastomer material is situated in part between the individual threads. It may also be provided that the elastomer material is situated in part between the individual threads. For example, the elastomer material, for example an LSR compound, may flow around the individual threads during production and may penetrate the gaps between them. A stable anchoring, with particularly high tensile strength, of the braid in the elastomer material may hereby be formed.

In accordance with one variant, the braid, together with the elastomer material, may form a fluid-tight tube wall of the tensile-strength-enhancing tube. This may be achieved again, for example, with silicone as elastomer material. By means of the fluid-tight tube wall, the tensile-strength-enhancing tube at the same time performs an insulating function, since it prevents the infiltration of blood into the interior of the electrode lead.

In accordance with a further variant, the tensile-strength-enhancing tube has a punched-out portion or a short cut at least over part of its length in order to facilitate the assembly. An opening in the form of a punched-out portion or a short cut is used to guide through one or more electrical conductors from the interior of the tensile-strength-enhancing tube outwardly. This facilitates the contacting of the electrodes arranged outside the tensile-strength-enhancing tube by electrical conductors arranged inside the tensile-strength-enhancing tube. An axial longitudinal cut which extends along the entire length of the tensile-strength-enhancing tube is used to assemble the tensile-strength-enhancing tube over longer components.

In accordance with a second aspect of the present invention, at least the object is also achieved by an implantable electrode lead which comprises a tensile-strength-enhancing tube in accordance with the first aspect of the present invention. For example, the electrode lead is intended for heart therapy, for example cardiac resynchronization therapy (CRT) or for neurostimulation (for example spinal cord stimulation). The electrode lead, in accordance with a preferred variant, may have such a tensile-strength-enhancing tube at least in a distal portion. For example, the tensile-strength-enhancing tube may undertake the majority of the tensile force transmission at least in the distal region of an electrode lead of this kind.

In accordance with one variant, an implantable electrode lead of this kind comprises a coradial coil, which extends within the tensile-strength-enhancing tube at least in some sections. The tensile-strength-enhancing tube may electrically insulate the coradial coil outwardly at least in some sections.

It is also included within the scope of the present invention that an implantable electrode lead of this kind comprises one or more ring electrodes, wherein the tensile-strength-enhancing tube may be provided in particular at least in the region of the at least one ring electrode. The tensile-strength-enhancing tube may extend here, for example, through the at least one ring electrode. Advantageously, an increase in the flexibility of the electrode lead in the region of the one or more ring electrodes or in the region of axially closely spaced-apart ring electrodes (for example dipole electrodes) may hereby be achieved. This is important at the time of implantation for the navigation, for example, of a CRT electrode through vessel branches as far as the target vein.

On the whole, the tensile-strength-enhancing tube according to the present invention allows a comparatively more economical production of an implantable electrode lead by a simple assembly which requires few components and few assembly steps, in particular, few adhesively bonded connections. In addition, a partly automated production of the components according to the present invention is possible, which in particular further reduces the number of necessary manual manufacturing steps. Corresponding products are furthermore characterized, in addition to the desired tensile strength enhancement with high bending flexibility and low extensibility, also by a high production reliability, because the tensile-strength-enhancing tube according to the present invention may also provide good stability with uniform quality, also in extremely critical areas.

A third aspect of the present invention provides a catheter that comprises a tensile-strength-enhancing tube in accordance with the first aspect of the present invention. In the case of catheters a tensile-strength-enhancing tube of this kind may bring similar advantages as those described in detail above with reference to implantable electrode leads. In particular, catheters with a smaller outer diameter benefit from the use of a tensile-strength-enhancing tube. Such catheters have an outer diameter of 8 F or less. In particular, with this technology, catheters with small outer diameters of less than 5 F may also be produced.

Additional features, aspects, objects, advantages, and possible applications of the present invention will become apparent from a study of the exemplary embodiments and examples described below, in combination with the Figures, and the appended claims

### DESCRIPTION OF THE DRAWINGS

Further advantages and embodiments of the present invention will be described hereinafter with reference to the figures, in which:

FIG. 1 shows an embodiment of a tensile-strength-enhancing tube according to the present invention with a cross thread and an axial thread;

FIG. 2 shows a further embodiment of a tensile-strength-enhancing tube according to the present invention with 8 cross threads and 4 axial threads;

FIG. 3 shows the braid belonging to the exemplary embodiment according to FIG. 2 on a braid core;

FIG. 4 shows the braid according to FIGS. 2 and 3 as a tailored segment;

FIGS. 5A-B shows a further embodiment of a tensile-strength-enhancing tube according to the present invention with a defined outer contour for the assembly of further components;

FIG. 6 shows an enlarged view of a multi-filament thread which may be used as cross thread and/or as axial thread;

FIG. 7 shows a further embodiment of a tensile-strength-enhancing tube according to the present invention which has a longitudinal cut in order to facilitate assembly;

FIG. 8 shows an embodiment of a CRT electrode lead according to the present invention with ring electrodes in the distal region; and

FIG. 9 shows a longitudinal section of an electrode lead according to the present invention with a coradial coil, in which a tensile-strength-enhancing tube according to the present invention extends through two ring electrodes.

### DETAILED DESCRIPTION

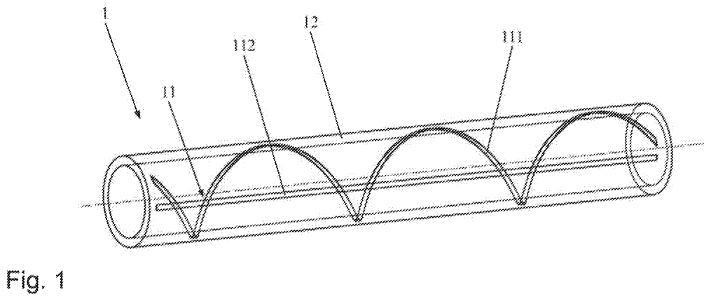

FIG. 1 shows schematically and by way of example an embodiment of a tensile-strength-enhancing tube 1 according to the present invention. The tensile-strength-enhancing tube 1 comprises a tubular braid 11, which is embedded in an elastomer material 12. In FIG. 1, the elastomer material 12, which defined the outer form of the tensile-strength-enhancing tube 1, is shown transparent so that the braid 11 is clearly visible.

In this exemplary embodiment, the braid 11 comprises a single cross thread 111, which extends helically along the tensile-strength-enhancing tube 1, and a single axial thread 112, which is woven with the cross thread 111 in such a way that the axial thread 112 is guided past intersection points with the cross thread 111 outside and inside the cross thread 111 alternately. The cross thread 111 and the axial thread 112 at the intersection points may additionally be fastened to one another by gluing or welding. The stability of the braid tube 11 as a whole may hereby be further increased.

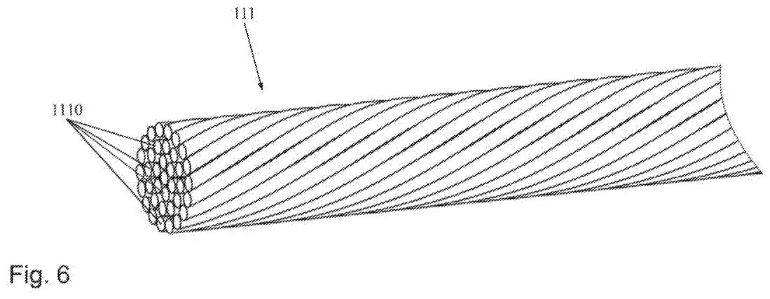

The cross thread 111 and the axial thread 112 are preferably made of a thermoplastic material, such as polyurethane (PU), polypropylene (PP), polyamide (PA), or polyethylene terephthalate (PET). The threads 111, 112 may be formed in particular as multi-filament threads, which are formed in each case of a plurality of individual threads 1110. This will be explained in greater detail further below with reference to FIG. 6.

The elastomer material 12 is an LSR silicone in the present example. In other words, the tensile-strength-enhancing tube 1 in this embodiment has been produced, for example, as an injection-molded part by an overmolding of the braid 11 with liquid silicone rubber (LSR compound). The use of silicone ensures a good flexibility of the tensile-force-transmitting tube 1 with respect to bending stress.

The axial thread 112 in the tensile-force-transmitting tube 1 fundamentally ensures the (axial) tensile force transmission. The cross thread 111 ensures a good anchoring of the axial thread 112 in the silicone and, in particular, prevents the axial thread 112 from being pulled out from the silicone 12 under tensile load.

The tensile-force-transmitting tube 1 in accordance with the present exemplary embodiment has an outer diameter of 1.0 mm with a wall thickness of 0.1 mm.

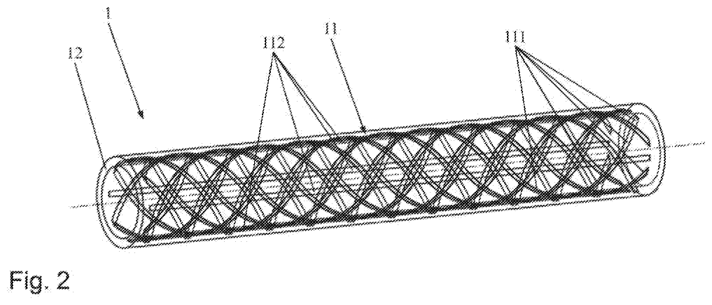

FIG. 2 shows schematically and by way of example further embodiments of a tensile-strength-enhancing tube 1 according to the present invention with a total of 8 cross threads 111 and 4 axial threads 112. Apart from the different number of threads, that said above with reference to FIG. 1 also applies for the variant according to FIG. 2.

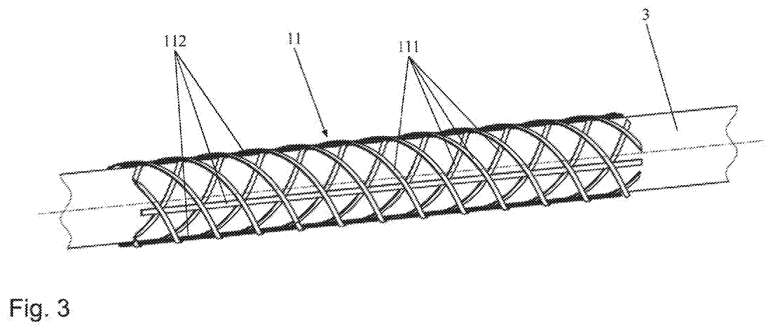

FIG. 3 shows schematically and by way of example the tri-axial braid 11, belonging to the exemplary embodiment according to FIG. 2, on a braid core 3.

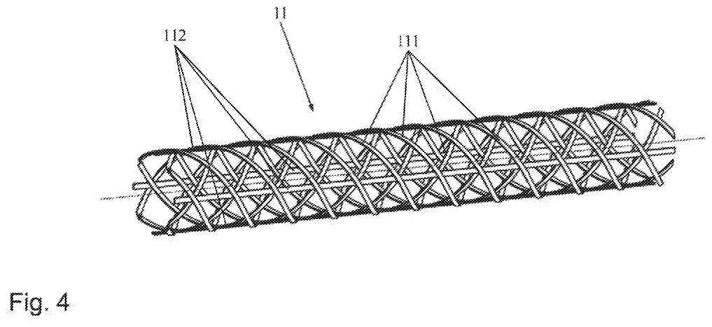

The structure of the braid 11 is shown particularly clearly on the basis of FIG. 4, which illustrates the braid 11 according to FIGS. 2 and 3 as a tailored segment in a simple perspective view. It is clear that 4 of the cross threads 111 of the braid 11 extend helically along the tensile-strength-enhancing tube 1 with a first direction of rotation (as “left-handed helix”), whereas the other 4 cross threads 111 extend helically with a second, opposite direction of rotation (i.e., as “right-handed helix”). The 4 axial threads 112 are arranged here uniformly (i.e., each distanced at 90° from one another) around the cross-section of the tensile-strength-enhancing tube 1. They are guided past the cross thread 111 in part inside and in part outside said cross thread. As already explained above in respect of the exemplary embodiment according to FIG. 1, the axial threads 111 and the cross threads 112 may additionally be adhesively bonded or welded at their intersection points, and the cross threads 112 may also be adhesively bonded or welded at their intersection points with themselves.

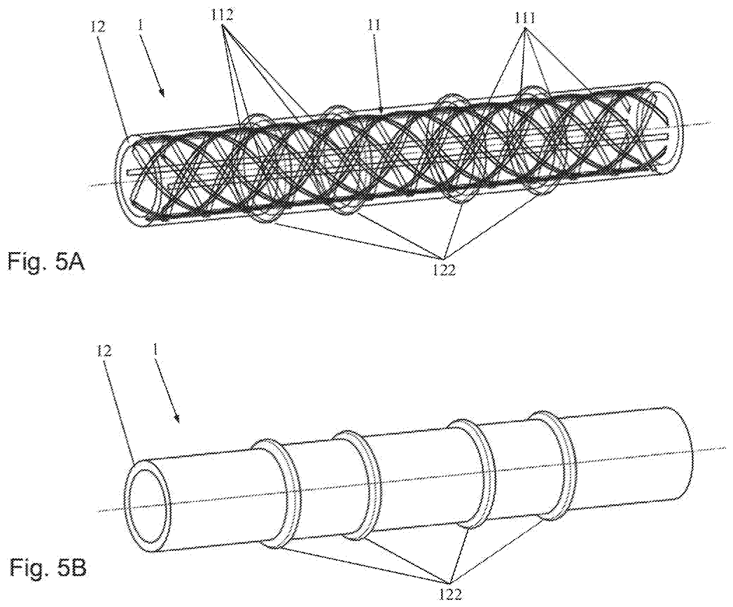

FIGS. 5A-B show a further variant which differs from the exemplary embodiment according to FIG. 2 merely by the form of the elastomer material 12. As shown in FIG. 5A, the braid 11 has the same structure as explained above with reference to FIGS. 2-4. However, in this exemplary embodiment, the tensile-strength-enhancing tube 1 has been manufactured as an injection-molded part, which has a defined outer contour, for example so as to allow the assembly of further components. This can be seen particularly well on the basis of FIG. 5B, in which the LSR silicone 12, which defines the outer contour of the tensile-strength-enhancing tube 1 is not shown transparent. Thus, the plurality of outer contour elements 122 are provided in the form of annular portions, in which the outer radius of the tensile-strength-enhancing tube 1 is increased. It is of course also conceivable that the tensile-strength-enhancing tube could be produced with defined inner contours (not illustrated).

FIG. 6 shows an enlarged view of a cross thread 11 as may be used in a tensile-strength-enhancing tube 1 according to the above-described exemplary embodiments. The cross thread 111 is embodied as a multi-filament thread from a number of individual threads 1110. A cross thread 111 is shown here by way of example, however, the one or more axial threads 112 according to the above-described exemplary embodiments may also be multi-filament threads of this kind. The shown multi-filament thread 111 is formed of a plurality of individual threads or individual filaments 1110, which may be stretched, woven or twisted. In the finished tensile-strength-enhancing tube 1, the LSR silicone 12 is preferably situated in part between the individual threads 1110. For example, during the production pf the tensile-strength-enhancing tube 1, the LSR compound 12 may flow around the individual threads 1110 and infiltrate the gaps between them. In other words the silicone compound may become positioned between the individual filaments 1110 during the overmolding of the braid 11. As a result of this mechanical anchoring, the threads 111, 112 may be prevented from being pulled out of the silicone. With use of multi-filament threads 111, 112, a stable anchoring, with particularly high tensile strength, of the braid 11 in the silicone 12 may thus be achieved.

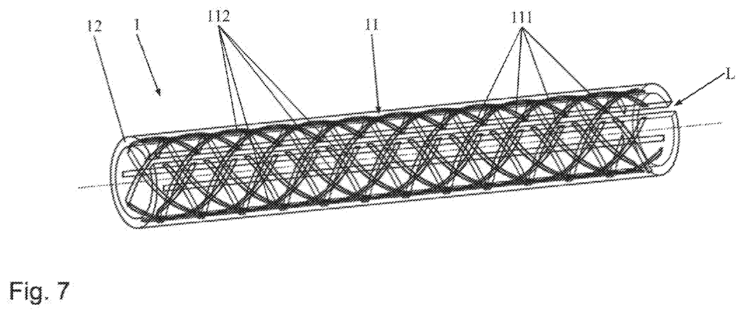

FIG. 7 shows schematically and by way of example a further embodiment of a tensile-strength-enhancing tube 1 according to the present invention. This differs from the exemplary embodiment according to FIG. 2 in that the tensile-strength-enhancing tube 1 has a longitudinal cut L to facilitate the assembly. The provision of such a longitudinal cut L may be advantageous for the assembly, since the braid tube 11 in this exemplary embodiment is substantially neither radially nor axially extensible.

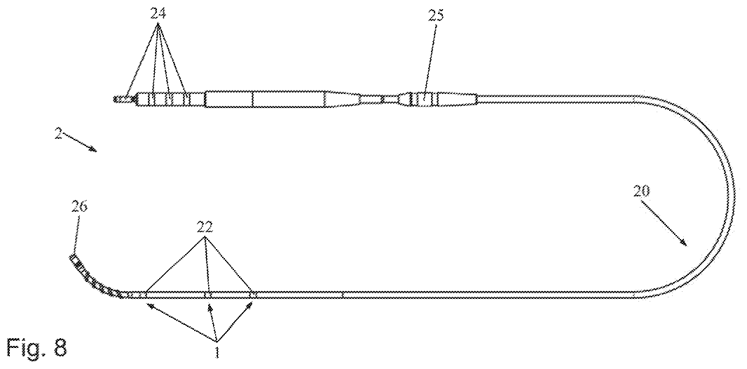

FIG. 8 shows an exemplary embodiment of an electrode lead 2 according to the present invention. The shown electrode lead 2 is intended for cardiac resynchronization therapy (CRT). It has an elongate lead body 20, wherein in a distal region a head electrode 26, and a plurality of ring electrodes 22 are arranged on the lead body 20. The electrodes 22, 26 are electrically active and are intended for the contacting of bodily tissue in the coronary sinus. FIG. 8 also shows an electrode fixing sleeve 25 and a plurality of plug contacts 24 for connection to a pulse emitter (not shown) in a proximal region of the electrode lead 2.

In the distal region of the electrode lead 2, which is to be introduced into the coronary sinus, the lead body 20 must be relatively flexible in respect of bending stresses and at the same time must be able to withstand the tensile forces occurring during implantation, repositioning and/or explantation. This is possible in the shown exemplary embodiment due to the provision of a tensile-force-transmitting tube 1 according to the present invention in the aforesaid distal region. The necessary tensile strength and at the same time the bending flexibility of the electrode lead 2 necessary for the application may hereby be ensured.

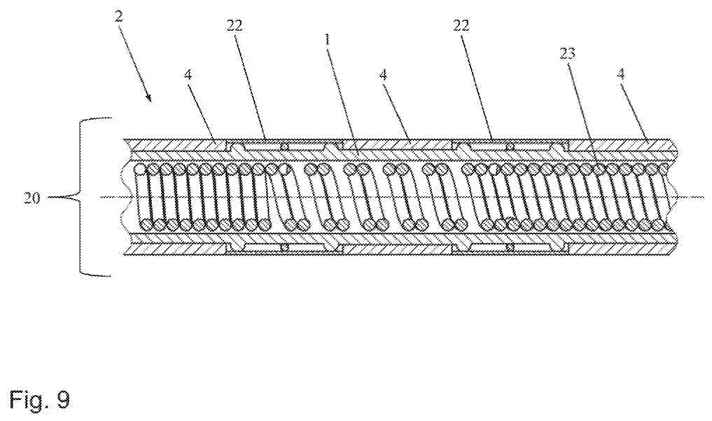

FIG. 9 shows a longitudinal cut of an electrode lead 2 according to the present invention in the region of a ring electrode 22. A conductive coradial coil 23 extends here within the tensile-force-transmitting tube 1 according to the present invention, which in the present case is shown merely schematically (i.e., without structural details of the braid 11). The tensile-force-transmitting tube 1 electrically insulates the coradial coil 3 and, by way of its fluid-tight design, additionally prevents the infiltration of blood into the interior of the lead body 20.

The tensile-force-transmitting tube 1 extends through the ring electrodes 22. By way of such an arrangement, the mechanical requirements in respect of tensile strength and flexibility with respect to bending stress may be satisfied, in particular also in the region of ring electrodes 22.

So that the electrodes 22, 26 do not protrude beyond the lead body 20 of the electrode lead 2, thus resulting in the creation of steps at the surface of the lead body 20 by the electrodes 22, 26, the tensile-force-transmitting tube 1 in FIG. 9 is encased by a cover tube 4 in the regions not enclosed by electrodes 22, 26. The cover tube 4 may be made, for example, from silicone or polyurethane.

In an alternative embodiment of an electrode lead 2 according to the present invention, instead of a conductive coradial coil 23 for electrical connection between the electrodes 22, 26 and the plug contacts 24, one or more conductive cables may also be used (not shown). The conductive cables extend here within the tensile-force-transmitting tube 1 of the electrode lead according to the invention. To guide the conductive cables and in order to insulate the conductive cables with respect to one another, a multi-lumen tube is provided within the tensile-force-transmitting tube 1. The aforementioned multi-lumen tube may advantageously be formed by the elastomer material 12 that is used to overmold the braid 11 with liquid silicone rubber (LSR compound) to form the tensile-strength-enhancing tube 1. A multi-lumen tube is understood to mean a tube in the interior of which a plurality of separate lumens extend from one end of the tube to the other end of the tube.

It will be apparent to those skilled in the art that numerous modifications and variations of the described examples and embodiments are possible in light of the above teachings of the disclosure. The disclosed examples and embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 tensile-strength-enhancing tube
11 braid
111 cross thread
112 axial thread
1110 individual threads
12 elastomer material
122 outer contour elements
2 implantable electrode lead
20 line body
22 ring electrode
23 coradial coil
24 plug contacts
25 electrode fixing sleeve
26 head electrode
3 braid core
4 cover tube
L longitudinal cut

I/We claim:

1. A tensile-force-enhancing tube for an implantable electrode lead or a catheter, comprising:
a tubular braid which is embedded in an elastomer material, wherein the braid comprises at least one cross thread and at least one axial thread.

2. The tensile-force-enhancing tube according to claim 1, wherein an outer diameter of the tensile-force-enhancing tube is less than or equal to 5 F.

3. The tensile-force-enhancing tube according to claim 1, wherein a wall thickness of the tensile-force-enhancing tube is less than or equal to 0.15 mm.

4. The tensile-force-enhancing tube according to claim 1, wherein the tubular braid comprises at least three cross threads.

5. The tensile-force-enhancing tube according to claim 1, wherein the elastomer material comprises a silicone.

6. The tensile-force-enhancing tube according to claim 1, wherein the at least one cross thread and/or the at least one axial thread comprises a thermoplastic material.

7. The tensile-force-enhancing tube according to claim 1, wherein the at least one cross thread and/or the at least one axial thread comprises polyurethane and/or polypropylene and/or polyamide and/or polyethylene terephthalate.

8. The tensile-force-enhancing tube according to claim 1, wherein the at least one cross thread and/or the at least one axial thread is a multi-filament thread formed from a plurality of individual threads.

9. The tensile-force-enhancing tube according to claim 8, wherein the elastomer material is situated in part between the individual threads of the at least one multi-filament thread.

10. The tensile-force-enhancing tube according to claim 1, wherein the tubular braid and the elastomer material form a fluid-tight tube wall.

11. An implantable electrode lead, which comprises a tensile-force-enhancing tube according to claim 1.

12. The implantable electrode lead according to claim 11, further comprising a coradial coil, which extends at least in some sections within the tensile-force-enhancing tube.

13. The implantable electrode lead according to claim 11, wherein the tensile-force-enhancing tube electrically insulated the coradial coil outwardly at least in some sections.

14. The implantable electrode lead according to claim 11, further comprising at least one ring electrode, wherein the tensile-force-enhancing tube extends through the at least one ring electrode.

15. A catheter, which comprises a tensile-force-enhancing tube according to claim 1.
